(19) United States
(12) Patent Application Publication
(10) Pub. No.: US 2005/0025735 A1
(43) Pub. Date: Feb. 3, 2005

(54) Method for time-dependent decrease of pH in a cosmetic composition and a composition for permanent hair shaping having time-dependent decreasing wave-shaping efficiency

(76) Inventors: David Rozzell, Burbank, CA (US); Juergen Allwohn, Burgschwalbach (DE); Thorsten Cassier, Dieburg (DE)

Correspondence Address: STRIKER, STRIKER & STENBY, 103 East Neck Road, Huntington, NY, 11743, US

(21) Appl. No.: 10/895,526

(22) Filed: Jul. 21, 2004

Related U.S. Application Data
(60) Provisional application No. 60/489,449, filed on Jul. 23, 2003.

(51) Int. Cl. A61K 7/06; A61K 7/11; A61K 7/09; A61K 38/46

(52) U.S. Cl. 424/70.14; 424/94.6

(57) ABSTRACT

A method for providing a time-dependent decrease of pH in a cosmetic composition is described. A method of permanently shaping hair is also described, in which, immediately prior to use, an enzyme that catalyzes an acid-generating reaction with a non-ester substrate is added to an alkaline composition, which contains the non-ester substrate, to form a ready-to-apply cosmetic composition. Then the hair, before and/or after it is put in a desired shape, is treated with the ready-to-apply cosmetic composition and, after a period of action, is rinsed from the hair with water. Thereafter the hair is oxidatively treated, rinsed again with water, optionally put in a water wave and dried. Preferably the enzyme and the non-ester substrate are selected so that the initial pH decreases by at least 0.5 pH units within a time interval of from 5 to 40 minutes.

### CROSS-REFERENCE

This application is based on U.S. Provisional Application 60/489,449, filed Jul. 23, 2003, and claims the benefit of priority based on this provisional application under 35 U.S.C. 119 (e).

### BACKGROUND OF THE INVENTION

1. Field of the Invention

The present invention relates to methods for time-dependent decrease of pH in a cosmetic composition and an agent for permanently shaping hair based on a keratin-reducing active ingredient, an enzyme that catalyzes an acid-generating reaction with a substrate and a substrate. The agent for permanently shaping hair has a time-dependent gradual reduction in waving effectiveness and a gentle shaping of hair with a high certainty of the result and improved care properties

2. Description of the Related Art

In general, the process of permanently shaping hair consists of two steps. Initially, the cystine disulfide bonds of the hair keratin are opened reductively by the action of an agent, which contains a keratin-reducing active ingredient. A transformation in which smooth hair is waved or curly hair is straightened, becomes possible as a result in that, after the reduction, the hair is rinsed and the cystine disulfide bonds are reestablished in their new position using an agent containing an oxidizing active ingredient.

As active reducing agents, mercaptocarboxylic acids are often used. In particular, thioglycolic acid or thiolactic acid or their salts, mercaptocarboxylate esters, cysteine and its derivatives or cysteamine and its derivates are especially useful.

The compositions containing thioglycolate are used usually at a pH between 8 and 10, and more preferably between a pH of 8.5 and 9.5. If this is done repeatedly, even at short intervals of time, the hair may be damaged.

A higher pH-value leads to an increased swelling of the hair as well as an increase in the reducing power of the mercapto compound used, leading to an increase in the effectiveness of the reducing step. Aside from the usual alkalizing agents, ammonia, ammonium hydrogen carbonate, ammonium carbonate, ammonium carbamate, monoethanolamine and diethanolamine are most commonly used for adjusting the pH to the alkaline range.

The above-mentioned mildly alkaline permanent waving preparations are applied in the usual manner on the hair. To achieve a permanent hair transformation, the agent must act for a certain period of time. Depending on the state of the treated hair, a prolonged period of action or a repeated application of the permanent waving preparation represents an increasing stress in the sense of an irreversible damage to the hair structure. In addition, it is a particular difficulty that the reducing process and the therewith associated softening of the hair proceed even after the prescribed period of action is reached, so that it is very important for the hairdresser to fix the correct end point. This is seen to be particularly relevant in the case of long hair, which has already been previously treated repeatedly with permanent waving agents, oxidative dyeing agents and/or bleaching agents. Since the treatment methods mentioned adversely affect the hair structure but damaged hair is particularly permeable for the shaping agents, excessive curling can result already when the optimum period of action is exceeded relatively slightly.

This problem, which occurs time and again in the daily practice of the hairdresser, has led to a series of attempted solutions, which are intended to counter the danger of damage to the hair during the permanent waving process by decreasing the waving effectiveness of the reducing agent during the period of action.

An agent and a method for continuously decreasing waving activity and consequential damage to the hair by lowering the initial pH are already known from German Patent Application DE 23 49 050 A. Organic compounds with ester or halogen groups in the molecule, which can be split by alkali and are capable of forming acid by the splitting, are added to the shaping agent shortly before use. By these means, the concentration of alkali is to decrease gradually and to the desired extent during the period of action. Ethyl acetate and triacetin have been named as examples of suitable esters. In the case of compounds containing ester groups, the splitting can be accelerated by also adding the lipase pancreatin.

Aside from the disadvantageous toxicology and skin compatibility of many of the substances mentioned, the esters also are too unstable in the presence of the shaping solution to be formulated with it. In addition, the low saponification rates of the recommended esters result in an insufficient release of acid during the period of action of 5 to 30 minutes. The enzyme pancreatin, mentioned in DE 23 49 050 A, accelerates the acid-generation from ester substrates, but pancreatin is insufficiently stable and looses activity rapidly in alkaline reducing solution. This is a significant disadvantage of the method of the prior art. When ester-splitting enzymes like pancreatin are used, stability problems make it necessary, for instance, to formulate the shaping agent in three components.

### SUMMARY OF THE INVENTION

An important advantage of the compositions and methods of the present invention is that a non-ester substrate is used. Such non-ester substrates are much more stable than ester-substrates in the presence of alkaline cosmetic compositions as described herein and undergo an acid-generating reaction only in the presence of a specific enzyme. A further advantage of the compositions and methods of the present invention is in the use of an enzyme that is more stable kept separately or even when contained in the alkaline composition than the ester-splitting enzymes as described in DE 23 49 050 A. In a preferred embodiment a hydantoinase is used as the enzyme. The hydatoinase that is the subject of the present invention has greater stability than ester splitting enzymes such as pancreatin that are described in the prior art, especially in the presence of alkaline reducing solutions such as those used in the permanent shaping of hair.

It is therefore an object of the invention to make available a cosmetic composition that comprises an enzyme that catalyzes an acid-generating reaction with a non-ester substrate, a method and an agent for the permanent shaping of hair, in which the pH drops rapidly enough during the period of action, so that the reducing power of the waving agent is weakened during the period of action and, by these means, on the one hand, the hair structure is treated more gently but, on the other, sufficient transformation of the hair is nevertheless achieved.

When the pH drops during the period of action the hair damage is reduced due to the decrease of the hair swelling and the weakened reducing power of the waving agent.

It has now surprisingly been found that the above-mentioned disadvantages of the method of the state of the art can be avoided by proceeding according to the present invention.

It is therefore an object of the invention to provide a cosmetic composition that comprises an enzyme that catalyzes an acid-generating reaction with a non-ester substrate.

As used herein, by “cosmetic composition” means a solution, a cream, a paste, an ointment or a suspension containing various ingredients and additives typically used in formulating a composition that is applied to the skin and/or the hair. Such ingredients and additives may include, but are not limited to, for example thickening agents, such as bentonite, fatty acids, starch, polyacrylic acid and its derivatives, cellulose derivatives, alginates, VASELINE®, paraffin oils; wetting agents or emulsifiers from the classes of anionic, cationic, amphoteric or nonionic surface-active substances, such as fatty alcohol sulfates, fatty alcohol ether sulfates, alkyl sulfonates, alkylbenzene sulfates, quaternary ammonium salts, alkylbetaines, ethoxylated alkylphenols, fatty acid alkanolamides or ethoxylated fatty esters; opacifiers, such as polyethylene glycol; glycol esters; alcohols, such as ethanol, propanol, isopropanol, polyols, such as ethylene glycol, 1,2- or 1,3-dihydroxypropane, 1,2-, 1,3- or 1,4-dihydroxybutane, 1,2-, 1,3-, 1,4-or 1,5-dihydroxy-pentane and glycerin; sugars, such as D-glucose; solubilizers; stabilizers; buffering substances; perfume oils; dyes as well as hair conditioning and hair care components, such as cationic polymers, lanolin, lanolin derivatives, cholesterol, pantothenic acid and betaine.

A further object of the invention is a cosmetic composition comprising an enzyme that catalyzes an acid-generating reaction with a non-ester substrate and a non-ester substrate.

In a preferred embodiment the enzyme has a half-life greater than 30 min in the presence of 10% by weight thioglycolate at pH 8.5.

In a preferred embodiment the non-ester substrate is a compound of formula (I) or 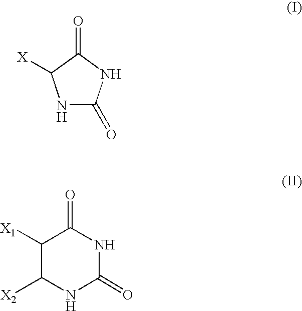
wherein X, X1 and X2 are each, independently of each other, selected from the group consisting of hydrogen, alkyl, aryl, arylalkyl and heterocyclic.

As used herein, “alkyl” means a straight chain or branched chain alkyl group having from 1 to 8 carbon atoms and in which 1 to 3 of the carbon atoms may optionally bear substituents selected from the group consisting of hydroxyl, alkoxy, amino, dialkylamino and carboxyl.

As used herein, “alkoxy” means a group consisting of an oxygen atom bound to an alkyl group as defined above.

As used herein, “dialkylamino” means a group consisting of a nitrogen atom bound to two alkyl groups as defined above.

As used herein, “aryl” means an aromatic ring containing 5 or 6 ring atoms which may be any combination of carbon, oxygen and nitrogen atoms, and which optionally may further contain from 1 to 3 substituents selected from the group consisting of hydroxy, alkoxy, amino, dialkylamino and carboxyl.

As used herein, “heterocyclic” means a carbon atom ring having 5 to 7 ring atoms further containing one or more atoms selected from the group consisting of oxygen, nitrogen and sulfur, and which optionally may further contain from 1 to 3 substituents selected from the group consisting of hydroxyl, alkoxy, amino, dialkylamino and carboxyl.

The present invention also comprises a method for time-dependent decrease of pH in a cosmetic composition for treating skin or hair, which includes the steps of:
(i) adding an enzyme that catalyzes an acid-generating reaction with a non-ester substrate to an alkaline composition having an initial pH of from about 7.0 to about 14 and comprising the non-ester substrate to form a ready-to-apply cosmetic composition;
(ii) treating the skin or hair of a person with the ready-to-apply cosmetic composition for a time sufficient to achieve the desired cosmetic effect.

In a preferred embodiment the enzyme and the substrate are selected so that the initial pH is from about 7.0 to about 12 and the initial pH is lowered within a time interval of from about 5 to about 40 minutes by at least about 0.5 pH units.

In other preferred embodiments of the composition and the method as described above the enzyme has a half-life of greater than 30 minutes in the presence of 10% by weight thioglycolate at a pH of 8.5.

In a further preferred embodiment of the method the adding of the enzyme is carried out immediately, within 10 minutes, and preferably within 10 seconds to 3 minutes, prior to use of the ready-to-apply cosmetic composition.

The method is preferably applied for time-dependent decrease of pH of a permanent hair shaping composition.

Thus another object of the invention is a method of permanently shaping hair, which comprises the steps of:
(a) adding an enzyme that catalyzes an acid-generating reaction with a non-ester substrate to an alkaline permanent shaping composition having an initial pH of from about 7.0 to about 14, which comprises a keratin-reducing active agent and the non-ester substrate, to form a ready-to-apply alkaline shaping agent immediately prior to treating the hair with the shaping agent;
(b) putting the hair in a desired shape;
(c) treating the hair with the ready-to-apply alkaline shaping agent for a period of action sufficient for permanent shaping of hair;
(d) after the period of action of the shaping agent, optionally rinsing the hair with water;
(e) after rinsing of the hair in step (d), oxidatively treating the hair and then optionally again rinsing the hair with water; and
(f) subsequently optionally putting the hair in a water wave and drying the hair.

Preferably the enzyme and the substrate are selected so that the initial pH is from about 7.0 to about 12 and is lowered within a time interval of from about 5 to about 40 minutes by at least about 0.5 pH units.

Preferably, the initial pH of the alkaline permanent shaping agent is from about 7.5 to about 10.0, more preferably from about 7.8 to about 9.5, and is decreased within about 10 to 30 minutes by about 0.5 to 3.0 units.

More preferably, the initial pH of the alkaline permanent shaping agent is from about 8.0 to about 10.0, and is decreased within about 10 to 30 minutes by about 1.0 to 2.0 units.

An initial pH of the permanent shaping agent of from 8.0 to 9.0 and a decrease of at least one pH unit to a pH of between 6.5 and 7.3 within 7 to 20 minutes are especially preferred.

The application temperature is preferably from 20° C. to 45° C. and more preferably 30° C. to 40° C.

In another preferred embodiment of the method the enzyme has a half-life of greater than 30 min in the presence of 10% by weight thioglycolate at a pH of 8.5.

The enzyme that catalyzes an acid-generating reaction with a non-ester substrate is preferably hydantoinase.

For carrying out the inventive method, it has proven to be particularly advantageous to use the enzyme in a concentration of 10 mg/l to 500 mg/l (0.01 to 0.50% by weight), and preferably of 20 mg/l to 200 mg/l (0.02 to 0.20% by weight) based on the ready-for-use permanent shaping agent.

The enzyme may be present in pure form or formulated as a mixture together with inert additives, such as perfume oils, wetting agents, solubilizers, dyes, etc., which are usually used in cosmetics. Preferably, the enzyme is dissolved in a non-aqueous solvent, optionally containing a multihydric alcohol, such as glycerin. In one preferred embodiment the enzyme is present in an aqueous ammonium sulfate suspension.

The non-ester substrate is preferably selected from the groups consisting of hydantoins and dihydrouracils, having the structures represented by formulas (I) or (II).

It has proven to be particularly advantageous to use the substrate in a concentration of 10 g/l to 100 g/l (1 to 10% by weight) and preferably of 20 g/l to 50 g/l (2 to 5% by weight), based on the ready-for-use permanent shaping agent.

In a further preferred embodiment of the method the adding of the enzyme is carried out immediately, within 10 minutes, and preferably within 10 seconds to 3 minutes prior to use of the ready-to-apply alkaline shaping agent.

The ready-to-apply permanent hair shaping agent may contain a substance to adjust the pH-value. By suitably selecting the concentration of the buffers, ammonium hydrogen carbonate, ammonium carbonate and/or ammonium carbamate, the pH of the permanent hair shaping agent and the amount of substrate and enzyme added, the decrease in the degree of dissociation of the reducing agent and, with that, the waving effectiveness during the period of action can be controlled to the desired degree by the decrease in the alkalinity. Preferably, a buffer, effective at a pH ranging from 6.8 to 7.5, is also added immediately before use. Aside from increasing the waving strength, the substances ammonium hydrogen carbonate, ammonium carbonate and ammonium carbamate have the task of buffering the pH towards the end of the period of action within the range of 6 to 7. Generally, the amount of buffer used ranges from 0.1 to 15% by weight and preferably from 1 to 8% by weight of the ready-for-use agent for permanently shaping hair.

The acid-generating reaction of the enzyme with the substrate can be accelerated by the heat usually supplied during permanent waving treatments.

As active, keratin-reducing agents, especially thioglycolic acid, thioglycolamide, thioglycerin, thiolactic acid, 3-mercaptopropionic acid, 2-mercaptopropionic acid, 3-mercaptopropionamide, 2-mercaptopropionamide, 2-mercaptoacetamide, cysteine, cysteamine, homocysteine, alkyl or acyl cysteines, or the salts or the derivatives of these compounds, especially ammonium thioglycolate, can be used. This active keratin-reducing agent is contained in the ready-for-use agent for the permanent shaping of hair in an amount of 1 to 25% by weight and preferably of 5 to 15% by weight.

Of course, the ready-for-use agent for the permanent shaping of hair may contain all additives, which are customary and known for the permanent shaping, for example, thickening agents, such as bentonite, fatty acids, starch, polyacrylic acid and its derivatives, cellulose derivatives, alginates, VASELINE®, paraffin oils; wetting agents or emulsifiers from the classes of anionic, cationic, amphoteric or nonionic surface-active substances, such as fatty alcohol sulfates, fatty alcohol ether sulfates, alkyl sulfonates, alkylbenzene sulfates, quaternary ammonium salts, alkylbetaines, ethoxylated alkylphenols, fatty acid alkanolamides or ethoxylated fatty esters; opacifiers, such as polyethylene glycol esters; alcohols, such as ethanol, propanol, isopropanol, polyols, such as ethylene glycol, 1,2- or 1,3-dihydroxy-propane, 1,2-, 1,3- or 1,4-dihydroxy-butane, 1,2-, 1,3-, 1,4- or 1,5-dihydroxy-pentane and glycerin; sugars, such as D-glucose; solubilizers; stabilizers; buffering substances; perfume oils; dyes as well as hair conditioning and hair care components, such as cationic polymers, lanolin, lanolin derivatives, cholesterol, pantothenic acid and betaine.

The components mentioned are used in the ready-for-use agent for the permanent shaping of hair in amounts customary for such purposes. For example, the wetting agents and emulsifiers are used in concentrations totaling 0.2 to 30% by weight, the alcohols in an amount totaling 0.1 to 20% by weight, the opacifiers, perfume oils and dyes in an amount of in each case of 0.1 to 1% by weight, the buffering substances in an amount totaling 0.1 to 10% by weight, the sugars, solubilizers, stabilizers, as well as hair conditioning and hair care components in an amount of in each case of 0.1 to 5% by weight, while the thickeners and solubilizers may be contained in this agent in an amount totaling 0.5 to 20% by weight.

Furthermore, to increase the effect, so-called swelling or penetrating materials, such as dipropylene glycol monomethyl ether, 2-pyrrolidone or imidazolidin-2-one may be contained in an amount of 1 to 30% by weight in the agent.

The ready-for-use agent for permanent shaping of hair is obtained by mixing several components. It is most advantageous if the ready-for-use agent is prepared by mixing two components immediately (5 seconds to 5 minutes, preferable 10 seconds to 3 minutes) before use, component (A) containing the keratin-reducing active ingredient and the substrate and component (B) containing the enzyme. The individual components may be present in solid or also in liquid or thickened form. Advantageously, the agent is manufactured as a two-component package.

In a further preferred embodiment the ready-for-use agent for the permanent shaping of hair is obtained by mixing two components immediately (5 seconds to 5 minutes, preferable 10 seconds to 3 minutes) before use, component (A) containing the keratin-reducing active ingredient in an aqueous formulation and component (B) containing the enzyme and the substrate in a solid form (powder or tablet).

In a further preferred embodiment of the inventive method for the permanent shaping of hair, the latter is treated as follows: Before the application, the enzyme of component (B) is added to a solution of component (A), which contains the mercapto compound and the substrate. The hair in curlers is now wetted in a known manner with the pre-shaping agent. The original pH of the mixture of preferably about 7.5 to 10 drops during the period of action to a value of from about 6.0 to about 7.5. The period of action depends on the extent of the curling desired and on the treatment temperature. After the treatment, the hair is given a subsequent oxidative treatment and then dried.

In a further preferred embodiment of the inventive method, the hair is washed initially with a shampoo and then rinsed with water. Subsequently, the hair, which has been toweled dry, is divided into individual strands and wound onto curlers with a diameter of 5 to 30 mm and preferably of 5 to 15 mm. Since, 10 seconds to 3 minutes before the application, the enzyme of the component (B) was mixed with the component (A) containing the hair keratin-reducing mercapto compound, the substrate and preferably a buffer substance, which is ammonium hydrogen carbonate, ammonium carbonate and/or ammonium carbamate, the hair is treated with an amount, sufficient for shaping the hair, preferably of 50 to 120 g of the ready-for-use shaping agent.

After a period of action, which is sufficient for the permanent shaping of the hair and which, depending on the nature of the hair, the shaping effectiveness of the shaping agent, as well as on the application temperature, amounts to 5 to 30 minutes (10 to 30 minutes without the action of heat or 5 to 20 minutes with the action of heat), the hair is rinsed with water and then given a subsequent oxidative treatment (“fixed”). Depending on the profusion of the hair, the agent for the subsequent oxidative treatment is preferably employed in an amount of 80 to 100 g.

For the subsequent oxidative treatment of the hair on curlers or not on curlers, any subsequent treatment agent, suitable for such a treatment, can be used. Examples of oxidizing agents, which can be used in such subsequent treatment agents, are potassium and sodium bromate, sodium perborate, urea peroxide and hydrogen peroxide. The concentration of the oxidizing agent varies, depending on the application time (usually 5 to 15 minutes) and on the application temperature. Usually, the oxidizing agent is present in the aqueous subsequent oxidative treatment agent in a concentration of 0.5 to 10% by weight. The agent for the subsequent oxidative treatment may, of course, contain other materials, such as wetting agents, care materials such as cationic polymers, weak acids, buffering substances or peroxide stabilizers and exist in the form of an aqueous solution or an emulsion as well as in a thickened form on an aqueous basis, especially as a cream, a gel or a paste. These conventional additives may be contained in the subsequent treatment agent, in particular, in an amount of 0.1 to 10% by weight.

Subsequently, the curlers are removed. If necessary, after the removal of the curlers, the hair can be treated once again oxidatively. The hair is then rinsed with water, optionally laid into a water wave and subsequently dried.

A further object of the invention is a new ready-to-apply permanent hair shaping agent having an initial pH of from about 7.0 to about 14.0, preferably from about of 7.5 to about 12.0, more preferably of from about 7.5 to about 10.0, and comprising a keratin-reducing active agent, a non-ester substrate and an enzyme that catalyzes an acid-generating reaction with the non-ester substrate.

A further object of the invention is a new ready-to-apply permanent shaping agent prepared by mixing an alkaline permanent shaping composition (A) having an initial pH of from about 7.0 to about 14.0, preferably from about of 7.5 to about 12.0, more preferably of from about 7.5 to about 10.0, and comprising a keratin-reducing active agent and a non-ester substrate, 5 seconds to 3 minutes prior to use with an enzyme (B) that catalyzes an acid-generating reaction with the substrate or with a composition containing the enzyme.

The agent and the method bring about an elastic, permanent and uniform shaping at the entire length from the hair regrowth (hair roots) up to the tip of the hair without producing allergic or sensitizing reactions under largely gentle conditions that do not damage the structure of the hair.

### EXAMPLES

### Example 1 Two-Component Hair Shaping Agent

| Component A | 14.8 | g | ammonium thioglycolate, 70% aqueous |
|---|---|---|---|
| | | | solution |
| | 2.6 | g | ammonium hydrogen carbonate |
| | 1.2 | g | 5,6-dihydrouracil |
| | 0.3 | g | dimethyldiallylammonium chloride |
| | | | homopolymer |
| | | | (CTFA: polyquaternium-6) |
| | 2.0 | g | glycerin polyethylene glycol ricinoleate |
| | | | (CTFA: PEG-35 castor oil) |
| | 1.0 | g | perfume oil |
| | to pH 8.4 | | ammonia |
| | ad 100.0 | g | water |
| Component B | 10.0 | g | aqueous ammonium sulfate suspension |
| | | | of 50 mg/lhydantoinase enzyme |

Before use, 100 g of component A and 1 g of component B are mixed together.

The washed, toweled dry hair, is wound onto curlers with a diameter of 6 mm and then moistened uniformly with the hair shaping agent, which is described above and was prepared one minute before use. The initial pH of 8.4 gradually decreases to 8.17 (a change of 0.23 units) after a period of action of 5 minutes. After a further period of action of 15 minutes at a pH of 8.17 at room temperature, the hair is rinsed thoroughly with water and then treated oxidatively with 80 g of a 3% aqueous hydrogen peroxide solution. After removal of the curlers, the hair is rinsed once more with water, laid into a water wave and subsequently dried. The hair, so treated, has a uniform and lively permanent wave.

### Example 2

### Two-Component Hair Shaping Agent

| Component | 14.5 | g | ammonium thioglycolate, 70% aqueous |
|---|---|---|---|
| A | | | solution |
| | 2.0 | g | thiolactic acid |
| | 2.0 | g | ammonium carbonate |
| | 2.4 | g | 5,6-dihydrouracil |
| | 0.7 | g | vinylpyrrolidone/ |
| | | | methacrylamidopropyltrimethylammonium |
| | | | chloride copolymer |
| | | | (CTFA: polyquaternium-28) |
| | 1.0 | g | hydrogenated, ethoxylated castor oil |
| | | | (CTFA: PEG-40 hydrogenated castor oil) |
| | 0.5 | g | perfume oil |
| | to pH 8.48 | | ammonia |
| | ad 100.0 | g | water |
| Component | 10.0 | g | aqueous ammonium sulfate suspension |
| B | | | of 50 mg/l hydantoinase enzyme |

Before use, 100 g of component A and 2 g of component B are mixed together.

The washed, toweled dry hair, is wound onto curlers with a diameter of 6 mm and then moistened uniformly with the hair shaping agent, which is described above and was prepared one minute before use. The initial pH of 8.48 gradually decreases to 7.66 (a change of 0.82 units) during a period of action of 5 minutes. After the whole period of action of 10 minutes at room temperature, the hair is rinsed thoroughly with water and then treated oxidatively with 80 g of a 3% aqueous hydrogen peroxide solution. After removal of the curlers, the hair is rinsed once more with water, laid into a water wave and subsequently dried. The hair, so treated, has a uniform and lively permanent wave.

### Example 3

### Two-Component Hair Shaping Agent

| Component A | 11.0 | g | ammonium thioglycolate, 70% aqueous |
|---|---|---|---|
| | | | solution |
| | 7.0 | g | cysteine |
| | 2.6 | g | ammonium hydrogen carbonate |
| | 4.8 | g | 5,6-dihydrouracil |
| | 0.7 | g | vinylpyrrolidone/N,N- |
| | | | dimethylaminoethylmethacrylate |
| | | | copolymer |
| | | | (CTFA: polyquaternium-11) |
| | 1.0 | g | polyoxyethylene (4) lauryl ether |
| | | | (CTFA: laureth-4) |
| | 0.5 | g | perfume oil |
| | to pH 8.48 | | ammonia |
| | ad 100.0 | g | water |
| Component B | 10.0 | g | aqueous ammonium sulfate suspension |
| | | | of 50 mg/l hydantoinase enzyme |

Before use, 100 g of component A and 2 g of component B are mixed together.

The washed, toweled dry hair, is wound onto curlers with a diameter of 6 mm and then moistened uniformly with the hair shaping agent, which is described above and was prepared one minute before use. The initial pH of 8.48 gradually decreases to 6.44 (a change of 2.04 units) during a period of action of 8 minutes. After the whole period of action of 15 minutes at room temperature, the hair is rinsed thoroughly with water and then treated oxidatively with 80 g of a 3% aqueous hydrogen peroxide solution. After removal of the curlers, the hair is rinsed once more with water, laid into a water wave and subsequently dried. The hair, so treated, has a uniform and lively permanent wave.

### Example 4

### Two-Component Hair Shaping Agent

| Component | 11.2 | g | ammonium thioglycolate, 70% aqueous |
|---|---|---|---|
| A | | | solution |
| | 5.3 | g | ammonium thiolactate, 50% aqueous |
| | | | solution |
| | 2.0 | g | ammonium hydrogen carbonate |
| | 4.8 | g | 5,6-dihydrouracil |
| | 1.7 | g | polyoxyethylene (4) lauryl ether |
| | | | (CTFA: laureth-4) |
| | 1.5 | g | vinylpyrrolidone/ |
| | | | methacrylamidopropyltrimethylammonium |
| | | | chloride copolymer |
| | | | (CTFA: polyquaternium-28) |
| | 0.5 | g | perfume oil |
| | to pH 8.40 | | ammonia |
| | ad 100.0 | g | water |
| Component | 10.0 | g | aqueous ammonium sulfate suspension |
| B | | | of 50 mg/l hydantoinase enzyme |

Before use, 100 g of component A and 0.4 g of component B are mixed together.

The washed, toweled dry hair, is wound onto curlers with a diameter of 6 mm and then moistened uniformly with the hair shaping agent, which is described above and was prepared one minute before use. The initial pH of 8.40 gradually decreases to 7.30 (a change of 1.1 units) after a period of action of 30 minutes. After the period of action of 30 minutes at room temperature, the hair is rinsed thoroughly with water and then treated oxidatively with 80 g of a 3% aqueous hydrogen peroxide solution. After removal of the curlers, the hair is rinsed once more with water, laid into a water wave and subsequently dried. The hair, so treated, has a uniform and lively permanent wave.

### Example 5

### Two-Component Hair Shaping Agent

| Component A | 14.8 | g | ammonium thioglycolate, 70% aqueous |
|---|---|---|---|
| | | | solution |
| | 2.0 | g | ammonium hydrogen carbonate |
| | 1.1 | g | hydantoin |
| | 0.6 | g | vinylpyrrolidone/methyl vinyl |
| | | | imidazolium chloride |
| | | | copolymer (CTFA: polyquaternium-16) |
| | 1.9 | g | coconut fatty alcohol polyoxyethylene |
| | | | ether (10 EO) (CTFA: coceth-10) |
| | 0.5 | g | perfume oil |
| | to pH 8.2 | | ammonia |
| | ad 100.0 | g | water |

| | |
|---|---|
| Component B | Mixture of 1 g starch powder and 0.05 mg hydantoinase |
| | enzyme (powder, lyophilisate). |

Before use component A and component B are mixed together.

The washed, toweled dry hair, is wound onto curlers with a diameter of 6 mm and then moistened uniformly with the hair shaping agent, which is described above and was prepared one minute before use. The initial pH of 8.40 gradually decreases to 7.30 (a change of 1.1 units) after a period of action of 30 minutes. After this period of action of 30 minutes at room temperature, the hair is rinsed thoroughly with water and then treated oxidatively with 80 g of a 3% aqueous hydrogen peroxide solution. After removal of the curlers, the hair is rinsed once more with water, laid into a water wave and subsequently dried. The hair, so treated, has a uniform and lively permanent wave.

### Example 6

### Two-Component Hair Shaping Agent

| Component A | 11.2 | g | ammonium thioglycolate, 70% aqueous |
|---|---|---|---|
| | | | solution |
| | 6.0 | g | ammonium thiolactate, 50% aqueous |
| | | | solution |
| | 2.0 | g | ammonium hydrogen carbonate |
| | 2.2 | g | hydantoin |
| | 0.7 | g | vinylpyrrolidone/N,N- |
| | | | dimethylaminoethylmethacrylate |
| | | | copolymer |
| | | | (CTFA: polyquaternium-11) |
| | 2.0 | g | polyoxyethylene (4) lauryl ether |
| | | | (CTFA: laureth-4) |
| | 0.5 | g | perfume oil |
| | to pH 8.2 | | ammonia |
| | ad 100.0 | g | water |
| Component B | 10.0 | g | aqueous ammonium sulfate suspension |
| | | | of 50 mg/l hydantoinase enzyme |

Before use, 100 g of component A and 1 g of component B are mixed together.

The washed, toweled dry hair, is wound onto curlers with a diameter of 6 mm and then moistened uniformly with the hair shaping agent, which is described above and was prepared one minute before use. The initial pH of 8.3 gradually decreases to 7.3 (a change of 1 pH unit) during a period of action of 30 minutes. After this period of action of 30 minutes at room temperature, the hair is rinsed thoroughly with water and then treated oxidatively with 80 g of a 3% aqueous hydrogen peroxide solution. After removal of the curlers, the hair is rinsed once more with water, laid into a water wave and subsequently dried. The hair, so treated, has a uniform and lively permanent wave.

### Example 7 to 24 Two-Component Hair Shaping Agents

In a base composition according to example 6 the hydantoin was replaced by the following amounts of hydantoin or dihydrouracil derivatives and applied to the hair accordingly. The time-dependent decrease of the pH is tabulated in the 4^{th} and 5^{th} columns in Table I below.

TABLE I
TIME-DEPENDENT DECREASE IN
pH of ALKALINE PERMANENT
SHAPING COMPOSITIONS ACCORDING
TO THE INVENTION

| Exam- | | | Initial | Final | Treating |
|---|---|---|---|---|---|
| ple | Substrate | Amount | pH | pH | time |
| 7 | 5-hydroxymethyl-hydantoin | 1.5 g | 8.3 | 7.8 | 30 min |
| 8 | 5-hydroxymethyl-hydantoin | 3.0 g | 8.3 | 7.2 | 25 min |
| 9 | 5-hydroxymethyl-hydantoin | 6.0 g | 8.3 | 6.5 | 20 min |
| 10 | 5-(2-hydroxyethyl)-hydantoin | 1.6 g | 8.3 | 7.3 | 30 min |
| 11 | 5-(2-hydroxyethyl)-hydantoin | 3.2 g | 8.3 | 7.0 | 27 min |
| 12 | 5-(2-hydroxyethyl)-hydantoin | 6.4 g | 8.3 | 6.5 | 20 min |
| 13 | 5-(2-carboxyethyl)-hydantoin | 1.8 g | 8.3 | 7.3 | 30 min |
| 14 | 5-(2-carboxyethyl)-hydantoin | 3.6 g | 8.3 | 6.8 | 25 min |
| 15 | 5-(2-carboxyethyl)-hydantoin | 7.2 g | 8.3 | 6.4 | 20 min |
| 16 | 5-(2-methoxyethyl)-hydantoin | 1.6 g | 8.3 | 7.3 | 30 min |
| 17 | 5-(2-methoxyethyl)-hydantoin | 3.2 g | 8.3 | 7.1 | 25 min |
| 18 | 5-(2-methoxyethyl)-hydantoin | 6.4 g | 8.3 | 6.5 | 20 min |
| 19 | 5-(2-dimethylaminoethyl)- | 2.0 g | 8.3 | 7.1 | 30 min |
| | hydantoin | | | | |
| 20 | 5-(2-dimethylaminoethyl)- | 4.0 g | 8.3 | 6.9 | 25 min |
| | hydantoin | | | | |
| 21 | 5-(2-dimethylaminoethyl)- | 6.0 g | 8.3 | 6.7 | 20 min |
| | hydantoin | | | | |
| 22 | 5-(2-hydroxyethyl)- | 1.8 g | 8.3 | 7.2 | 30 min |
| | dihydrouracil | | | | |
| 23 | 5-(2-hydroxyethyl)- | 3.6 g | 8.3 | 6.8 | 25 min |
| | dihydrouracil | | | | |
| 24 | 5-(2-hydroxyethyl)- | 7.2 g | 8.3 | 6.4 | 20 min |
| | dihydrouracil | | | | |

While the invention has been illustrated and described as embodied in a method for time-dependent decrease of pH in a cosmetic composition and a composition for permanent hair shaping having time-dependent decreasing wave-shaping efficiency, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A cosmetic composition comprising at least one cosmetic ingredient and an enzyme that catalyzes an acid-generating reaction of a non-ester substrate.

2. The cosmetic composition as defined in claim 1, further comprising a non-ester substrate for said enzyme.

3. A cosmetic composition comprising at least one cosmetic ingredient, a non-ester substrate and an enzyme that catalyzes an acid-generating reaction of said non-ester substrate;
wherein said non-ester substrate is a compound of formula (I) or (II): 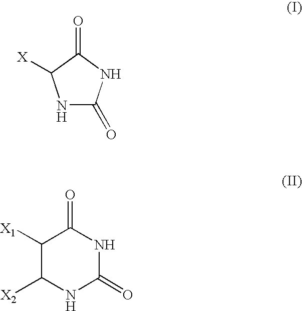
wherein X, X1 and X2 are, independently of each other, each selected from the group consisting of hydrogen, alkyl, aryl, arylalkyl and heterocyclic.

4. The composition as defined in claim 1, 2 or 3, wherein said enzyme has a half-life greater than 30 min at pH 8.5 in the presence of 10% by weight thioglycolate.

5. The composition as defined in claim 1, 2 or 3, wherein the at least one cosmetic ingredient is selected from the group consisting of thickening agents, anionic surface-active substances, cationic surface-active substances, amphoteric surface-active substances, nonionic surface-active substances, opacifiers, alcohols, sugars, solubilizers, stabilizers, buffering substances, perfume oils, dyes, hair conditioning ingredients and hair care ingredients.

6. The composition as defined in claim 1, 2 or 3, wherein the at least one cosmetic ingredient is selected from the group consisting of bentonite, fatty acids, starch, polyacrylic acid, cellulose compounds, alginates, petrolatum, paraffin oils, fatty alcohol sulfates, fatty alcohol ether sulfates, alkylsulfonates, alkylbenzene sulfates, quaternary ammonium salts, alkylbetaines, ethoxylated alkylphenols, fatty acid alkanolamides, ethoxylated fatty esters, polyethylene glycol, esters, ethanol, propanol, isopropanol, ethylene glycol, 1,2-dihydroxy-propane, 1,3-dihydroxypropane, 1,2-dihydroxybutane, 1,3-dihydroxybutane, 1,4-dihydroxybutane, 1,2-dihydroxypentane, 1,3-dihydroxypentane, 1,4-dihydroxypentane, 1,5-dihydroxypentane, glycerol, D-glucose, solubilizers, stabilizers, buffering substances, perfume oils, dyes, cationic polymers, silicone polymers, cationic silicone polymers, lanolin, lanolin derivatives, cholesterol, pantothenic acid and betaine.

7. A method for time-dependent decrease of pH in a cosmetic composition for treating skin or hair of a person, said method comprising the steps of:
a) adding an enzyme that catalyzes an acid-generating reaction of a non-ester substrate to an alkaline composition having an initial pH of from about 7.0 to about 14, said alkaline composition comprising said non-ester substrate, to form a ready-to-apply cosmetic preparation; and
b) treating the skin or the hair of the person with said ready-to-apply cosmetic preparation for a time period sufficient to achieve a desired cosmetic effect.

8. The method as defined in claim 7, wherein said enzyme and said non-ester substrate are selected so that said initial pH is from about 7.0 to about 12 and is lowered by at least 0.5 pH units within a time interval of from 5 to 40 minutes.

9. The method as defined in claim 7, wherein said enzyme has a half-life greater than 30 min at pH 8.5 in the presence of 10% by weight of thioglycolate.

10. A method for permanently shaping hair, said method comprising the steps of:
(a) adding an enzyme that catalyzes an acid-generating reaction of a non-ester substrate to an alkaline permanent shaping composition having an initial pH of 7.0 to 14, said permanent shaping composition comprising a keratin-reducing active agent and a non-ester substrate, immediately prior to permanently shaping the hair, to form a ready-to-apply alkaline shaping agent;
(b) putting the hair in a desired shape;
(c) treating the hair with said ready-to-apply shaping agent for a period of action sufficient for permanent shaping of hair;
(d) after the period of action of said shaping agent, optionally rinsing the hair with water;
(e) after the rinsing of the hair in step (d), oxidatively treating the hair and then optionally again rinsing the hair with water; and
(f) subsequently optionally putting the hair in a water wave and drying the hair.

11. The method as defined in claim 10, wherein the enzyme and the non-ester substrate are selected, so that said initial pH is from 7.0 to 12 and is lowered by at least 0.5 pH units within a time interval of from 5 to 40 minutes.

12. The method as defined in claim 10, wherein said enzyme and said non-ester substrate are selected, so that said initial pH is from 7.5 to 10 and is lowered by about 0.5 to about 3.0 pH units within from 10 to 30 minutes.

13. The method as defined in claim 10, 11 or 12, wherein said enzyme is hydantoinase.

14. The method as defined in claim 10, 11 or 12, wherein said non-ester substrate is a hydantoin or a dihydrouracil of formula (I) or (II): 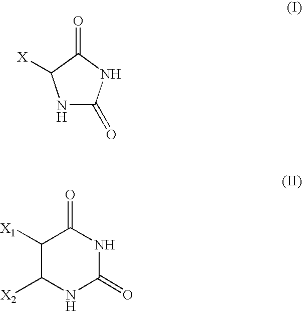
wherein X, X1 and X2 are, independently of each other, each selected from the group consisting of hydrogen, alkyl, aryl, arylalkyl and heterocyclic.

15. The method as defined in claim 10, 11 or 12, wherein said initial pH is from 7.8 to 9.5.

16. The method as defined in claim 10, 11 or 12, wherein said initial pH is from 8.0 to 9.0.

17. The method as defined in claim 10, 11 or 12, wherein said enzyme is added to said alkaline permanent shaping composition at a time of from 10 seconds to 3 minutes prior to application of the ready-to-apply alkaline shaping agent to the hair.

18. A ready-to-apply permanent hair shaping agent prepared 5 seconds to 3 minutes prior to use by mixing an alkaline permanent shaping composition having an initial pH of 7.0 to 14 and comprising a keratin-reducing active agent and a non-ester substrate with an enzyme that catalyzes an acid-generating reaction with said non-ester substrate or a composition containing said enzyme that catalyzes said acid-generating reaction with said non-ester substrate.

19. The ready-to-apply permanent hair shaping agent as defined in claim 18, wherein said initial pH is from about 7.0 to about 12.0.

20. The ready-to-apply permanent hair shaping agent as defined in claim 18, wherein said initial pH is from about 7.5 to about 10.0.

21. The ready-to-apply permanent hair shaping agent as defined in claim 18, wherein said enzyme has a half-life greater than 30 min at pH 8.5 in the presence of 10% by weight of thioglycolate.

22. The ready-to-apply permanent hair shaping agent as defined in claim 18, wherein said initial pH is from about 8.0 to about 9.0.

23. The ready-to-apply permanent hair shaping agent as defined in claim 18, wherein said enzyme is hydantoinase.

24. The ready-to-apply permanent hair shaping agent as defined in claim 18, containing from 0.01 to 0.50% by weight of said enzyme.

25. The ready-to-apply permanent hair shaping agent as defined in claim 18, wherein said non-ester substrate is a hydantoin or a dihydrouracil.

26. The ready-to-apply permanent hair shaping agent as defined in claim 18, containing from 1 to 10% by weight of said non-ester substrate.

27. The ready-to-apply permanent hair shaping agent as defined in claim 18, wherein the keratin-reducing active agent is a mercapto compound.

28. The ready-to-apply permanent hair shaping agent as defined in claim 18, wherein the keratin-reducing active agent is thioglycolic acid, thioglycerine, thioglycolamide, thiolactic acid, 3-mercaptoprpionic acid, cysteine, cysteamine, homocysteine, an alkyl cysteine or acyl cysteine, or a salt thereof.

29. The ready-to-apply permanent hair shaping agent as defined in claim 27 or 28, containing from 5 to 15% by weight of said keratin-reducing active agent.

30. A ready-to-apply permanent hair shaping agent having a pH of from about 7.0 to about 14.0 and comprising a keratin-reducing active agent, a non-ester substrate and an enzyme that catalyzes an acid-generating reaction with said non-ester substrate.

31. A ready-to-apply permanent hair shaping agent having an pH of from about 7.5 to about 12.0 and comprising a keratin-reducing active agent, a non-ester substrate and an enzyme that catalyzes an acid-generating reaction with said non-ester substrate.

32. The ready-to-apply permanent hair shaping agent as defined in claim 30 or 31, wherein said enzyme has a half-life greater than 30 min at pH 8.5 in the presence of 10% by weight thioglycolate.
